(19) United States
(12) Patent Application Publication
(10) Pub. No.: US 2012/0123544 A1
(43) Pub. Date: May 17, 2012

(54) Intervertebral Spacer and Method of Installation Thereof

(76) Inventors: Sean Suh, Plymouth Meeting, PA (US); David C. Paul, Phoenixville, PA (US); Damien O'Halloran, King of Prussia, PA (US)

(21) Appl. No.: 12/947,427

(22) Filed: Nov. 16, 2010

(51) Int. Cl. A61F 2/44

(52) U.S. Cl. 623/17.16

(57) ABSTRACT

The present invention provides an intervertebral spacer capable of being installed into an intervertebral disc space. In one embodiment, the intervertebral spacer comprises a body portion. The body portion comprises a first end, a second end, a first side portion connecting the first end and the second end, and a second side portion connecting the first end and the second end. An entrance port is defined in the first end of the body portion. A first exit port is defined in the first side portion of the body portion. A second exit port is defined in the second side portion of the body portion.

### FIELD OF THE INVENTION

The present invention relates to an apparatus and method for promoting an intervertebral fusion, and more particularly relates to an intervertebral spacer having one or more ports for delivery of a disc filler material into the disc space.

### BACKGROUND OF THE INVENTION

A common procedure for handling pain associated with intervertebral discs that have become degenerated due to various factors such as trauma or aging is the use of intervertebral spacers for fusing one or more adjacent vertebral bodies. Generally, to fuse the adjacent vertebral bodies, the intervertebral disc is first partially or fully removed. A spacer is then typically inserted between neighboring vertebrae to maintain normal disc spacing and restore spinal stability, thereby facilitating an intervertebral fusion.

There are a number of known conventional spacers and methodologies in the art for accomplishing the intervertebral fusion. These include screw and rod arrangements, solid bone implants, and fusion devices which include a cage or other implant mechanism which, typically, is packed with a filler material for promoting fusion. These spacers are implanted between adjacent vertebral bodies in order to fuse the vertebral bodies together, alleviating the associated pain. In some instances, the filler material can be introduced around and within the spacer to promote and facilitate the intervertebral fusion. For example, the spacer can be packed with the filler material to promote the growth of bone through and around the spacer. By way of further example, the filler material can be packed between the endplates of the adjacent vertebral bodies prior to, subsequent, or during implantation of the spacer. However, after placement of the spacer, introduction of the filler material into the surround space can be difficult.

As such, there exists a need for a spacer capable of being installed inside an intervertebral disc space through which disc filler material can be delivered into the disc space.

### SUMMARY OF THE INVENTION

In an exemplary embodiment, the present invention provides an intervertebral spacer capable of being installed into an intervertebral disc space. In one embodiment, the intervertebral spacer comprises a body portion. The body portion comprises a first end, a second end, a first side portion connecting the first end and the second end, and a second side portion connecting the first end and the second end. An entrance port is defined in the first end of the body portion. A first exit port is defined in the first side portion of the body portion. A second exit port is defined in the second side portion of the body portion.

In another exemplary embodiment, the present invention provides a method of accessing an intervertebral disc space. In one embodiment, the method comprises drilling an access channel to the intervertebral disc space through an adjacent vertebra. The adjacent vertebra comprising a pedicle and an endplate adjacent the intervertebral disc space. The access channel penetrates the pedicle and the endplate of the adjacent vertebra.

Further areas of applicability of the present invention will become apparent from the detailed description provided hereinafter. It should be understood that the detailed description and specific examples, while indicating the preferred or exemplary embodiments of the invention, are intended for purposes of illustration only and are not intended to limit the scope of the invention. Although individual embodiments are discussed, the invention covers all combinations of all those embodiments

### BRIEF DESCRIPTION OF THE DRAWINGS

The present invention will become more fully understood from the detailed description and the accompanying drawings, wherein:

FIG. 1 is a side view of an embodiment of a spacer shown between adjacent vertebrae according to the present invention;

FIG. 2 is a front perspective view of a spacer in accordance with one embodiment of the present invention;

FIG. 3 is a front end view of a spacer in accordance with one embodiment of the present invention;

FIG. 4 is a top view of a spacer in accordance with one embodiment of the present invention;

FIG. 5 is a side view of a spacer in accordance with one embodiment of the present invention;

FIG. 6 illustrates delivery of filler material through a spacer and into the intervertebral disc space, in accordance with one embodiment of the present invention;

FIG. 7 is a front perspective of a spacer in accordance with an alternative embodiment of the present invention;

FIG. 8 is a top view of a spacer in accordance with an alternative embodiment of the present invention;

FIG. 9 is a side view of the lumbar region of a patient's spine;

FIG. 10 illustrates insertion of a drill through a pedicle in accordance with one embodiment of the present invention;

FIGS. 11-12 illustrate a drill penetrating the superior endplate to form an access channel to the intervertebral disc space in accordance with one embodiment of the present invention;

FIG. 13 illustrates a balloon assembly in accordance with one embodiment of the present invention;

FIG. 14 illustrates insertion of a balloon into the intervertebral disc space using a trans pedicle-endplate approach in accordance with one embodiment of the present invention;

FIG. 15 illustrates inflation of a balloon in the intervertebral disc space in accordance with one embodiment of the present invention;

FIG. 16 illustrates a rod assembly that can be inserted into a channel formed in a vertebra in accordance with one embodiment of the present invention; and

FIG. 17 illustrates insertion of the rod assembly of FIG. 16 into a channel formed in a vertebra in accordance with one embodiment of the present invention.

### DETAILED DESCRIPTION OF THE PREFERRED EMBODIMENTS

The following description of the preferred embodiment(s) is merely exemplary in nature and is in no way intended to limit the invention, its application, or uses.

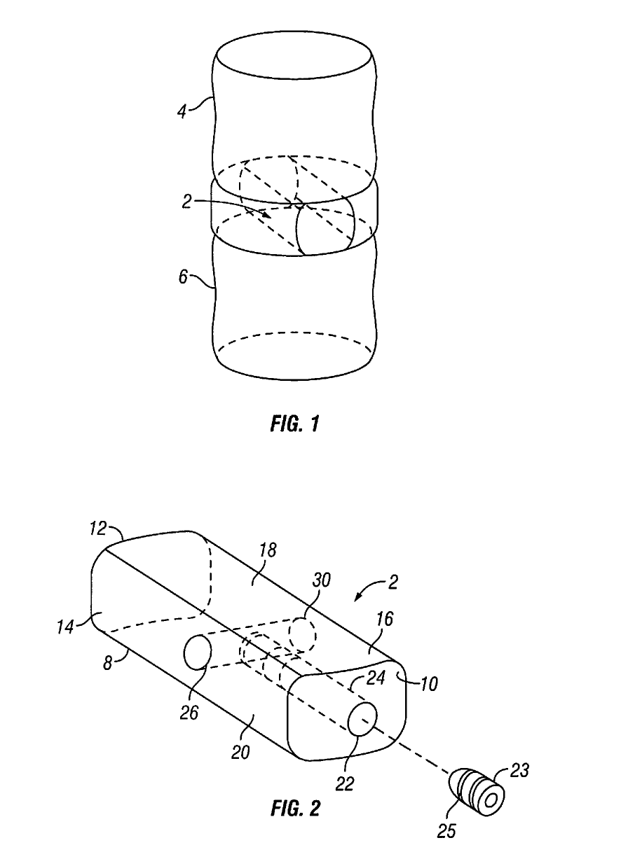

A spinal fusion is typically employed to eliminate pain caused by the motion of degenerated disk material. Upon successful fusion, a spacer becomes permanently fixed within the intervertebral disc space. Looking at FIG. 1, an exemplary embodiment of a spacer 2 is shown between adjacent vertebral bodies 4 and 6. In this position, the spacer 2 should help to maintain normal intervertebral disc spacing and restore spinal stability, thereby facilitating an intervertebral fusion.

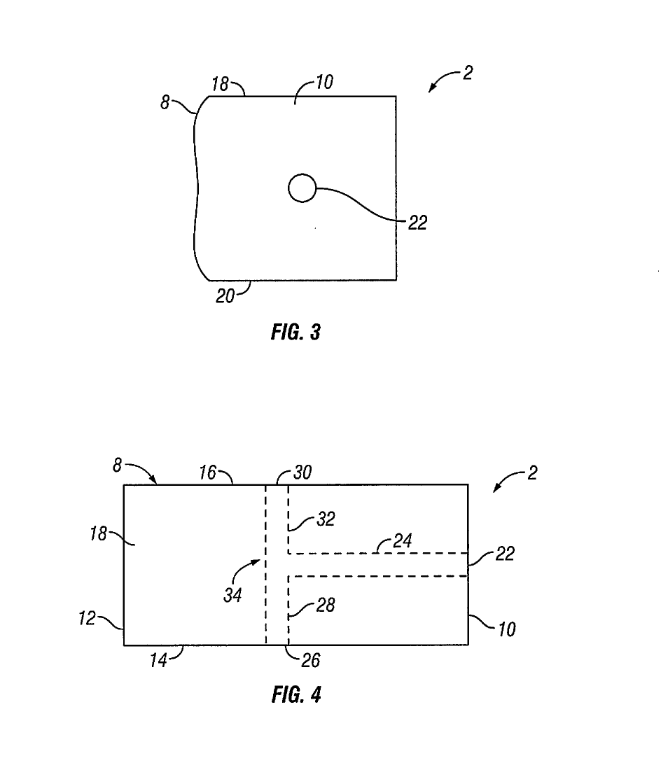

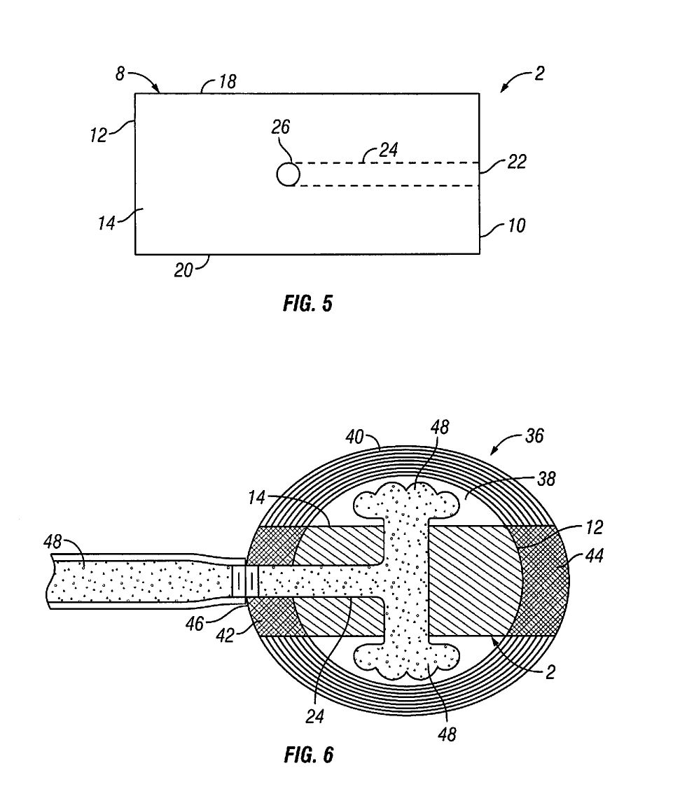

With reference to FIGS. 2-5, a spacer 2 is shown in accordance with one embodiment of the present invention. In an exemplary embodiment, the spacer 2 includes a body portion 8 having a first end 10, a second end 12, a first side portion 14 connecting the first end and the second end 12, and a second side portion 16 on the opposing side of the body portion 8 connecting the first end 10 and the second end 12. The body portion 8 further includes an upper surface 18 and a lower surface 20. While the body portion 8 can be configured to have any of a variety of different shapes, in an exemplary embodiment, the upper and lower surfaces 18, 20 of the body portion 8 may be generally rectangular in shape, as best seen in FIG. 4. In an embodiment, the body portion 8 may be generally block-shaped having six rectangular sides. In another embodiment (not illustrated), the upper and lower surfaces 18, 20 may be generally trapezoidal in shape. It should be understood that other shapes for the body portion 8 may also be suitable as desired for a particular application.

The first end 10 of the body portion 8, in an exemplary embodiment, includes an entrance port 22. In one embodiment, the entrance port 22 is in fluid communication with a channel 24 in the body portion 8. The entrance port 22 can be sized to receive the dispensing end of a syringe or other device for delivering disc filler material. In an embodiment, the cross-sectional area of the entrance port 22 may be about 0.5 mm² to about 4 mm². Typically, the cross-sectional area of the entrance port 22 is small in size when compared to the surface area of the first end 10. In an embodiment, the ratio of the cross-sectional area of the entrance port 22 to the surface area of the first end 10 is less than 1:10 and more preferably less than 1:20. The body portion 8 further may include a plug 23. The plug 23 may be configured and dimensioned for insertion into the entrance port 22 to seal the entrance port 22. In an embodiment, the plug 23 includes threading 25.

The first side portion 14 of the body portion 8, in an exemplary embodiment, includes a first exit port 26. In one embodiment, the first exit port 26 is in fluid communication with a first branch 28 of the channel 24 in the body portion 8. The first exit port 26 can be sized to deliver disc filler material into the disc space. In an embodiment, the cross-sectional area of the first exit port 26 may be about 0.5 mm² to about 4 mm². Typically, the cross-sectional area of the first exit port 26 is small in size when compared to the surface area of the first side portion 14. In an embodiment, the ratio of the cross-sectional area of the first exit port 26 to the surface area of the first side portion 14 is less than 1:10 and more preferably less than 1:20.

The second side portion 16 of the body portion 8, in an exemplary embodiment, includes a second exit port 30. In one embodiment, the second exit port 30 is in fluid communication with a second branch 32 of the channel 24 in the body portion 8. The second exit port 30 can be sized to deliver disc filler material into the disc space. In an embodiment, the cross-sectional area of the second exit port 30 may be about 0.5 mm² to about 4 mm². Typically, the cross-sectional area of the second exit port 30 is small in size when compared to the surface area of the second side portion 16. In an embodiment, the ratio of the cross-sectional area of the second exit port 30 to the surface area of the second side portion 16 is less than 1:10 and more preferably less than 1:20.

As best seen in FIG. 4, the body portion 8 includes the channel 24. In the illustrated embodiment, the channel 24 proceeds in the direction of the longitudinal axis of the body portion 8 until dividing into a first branch 28 and a second branch 32. In one embodiment, the division of the channel 24 occurs at junction 34. In an exemplary embodiment, the junction 34 is a t-shaped junction. In an exemplary embodiment, the first branch 28 is in fluid communication with the first exit port 26, and the second branch 32 is in fluid communication with the second exit port 30. In one embodiment, the channel 24 is a generally tubular-shaped channel. The first branch 28 and the second branch 32 can also be generally tubular-shaped channels in accordance with embodiments of the present invention. The channel 24 should generally be sized to deliver disc filler material from the entrance port 22 to the first exit port 26 and the second exit port 30. In an embodiment, the diameter of the channel 24 may be about 0.5 mm to about 4 mm.

Any of a variety of different biocompatible materials may be used to manufacture the spacer 2. By way of example, suitable materials may include titanium, stainless steel, titanium alloys, non-titanium metallic alloys, polymeric materials, plastics, plastic composites, polyetheretherketone (PEEK), ceramic, and elastic materials. In an embodiment, the body portion 8 can be manufactured from a material that comprises an elastomeric material. However, it should be understood that other materials may be used to manufacture all or part of the spacer 2.

An embodiment of method of installing the spacer 2 into the intervertebral disc space 36 is now discussed with reference to FIG. 6. Prior to insertion of the spacer 2, the intervertebral disc space 36 is prepared. The intervertebral disc space 36 may be prepared, for example, by complete or partial removal of the patient's disc. In the illustrated embodiment, the disc nucleus has been removed with the disc annulus 40 remaining at least partially intact. As illustrate by FIG. 6, removal of the disc nucleus forms a cavity 38 for insertion of the spacer 2. The endplates of the adjacent vertebral bodies 2, 3 (best seen on FIG. 1) can then scraped to create an exposed end surface for facilitating bone growth across the disc space 36. The spacer 2 is then introduced into the cavity 38, with the second end 12 of the body portion 8 being inserted first into the cavity 38 followed by the second end 14. First and second synthetic annulus materials 42, 44 may be placed on either end of the spacer 2. The first synthetic material 44 may have a through passageway 46 that is disposed in communication with the entrance port 22 (best seen on FIG. 4) of the spacer 2.

With the spacer 2 inserted into and seated in the appropriate position in the intervertebral disc space 36, the disc filler material 48 can then be injected into the disc space 36 through the spacer 2. To inject the filler material 48, a delivery device (not illustrated) may be used. The delivery device may include any of a variety of different devices suitable for delivering the filler material 48 into the disc space 36, including, for example, syringe-type devices and cement guns. As illustrated, the filler material 48 may be introduced into the disc space 36 from the delivery device through the passageway 46 in the first synthetic material 44 and the channel 24 in the spacer 2. In general, the filler material 48 can be introduced into the portion of the cavity 38 in the disc space 36 that is not occupied by the spacer 2. In an embodiment, the filler material 48 can fill the portion of the cavity 38 that is not occupied by the spacer 2. In another embodiment, the filler material 48 can partially fill the portion of the cavity 38 that is not occupied by the spacer 2.

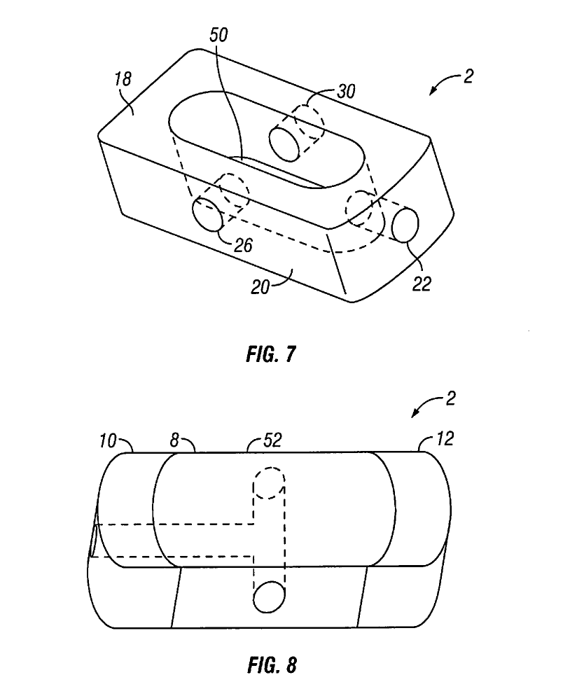

FIG. 7 illustrates an alternative embodiment of the spacer 2 illustrated by FIGS. 2-6. As illustrated, the spacer 2 includes a central cavity 50 which is in fluid communication with entrance port 22, first exit port 26, and second exit port 30. In an embodiment, the central cavity 50 is open on both the upper surface 22 and lower surface 20 of the spacer. In other words, the central cavity extends through the spacer 2 from the upper surface 22 to the lower surface 20. After introduction into of the spacer 2 into the disc space, filler material can be introduced into the central cavity 50 via the entrance port 22. When the central cavity 50 is filled, any additional material that is introduced should force filler material out from the exit ports 26, 30 and into the disc space.

FIG. 8 illustrates another alternative embodiment of the spacer 2 illustrated by FIGS. 2-6. As illustrated, the spacer 2 includes a body portion 8 having a first end 10 and a second end 12. In the illustrated embodiment, the body portion 8 includes a middle portion 52 between the first end 10 and the second end 12. In an embodiment, the first end 10 and the second end 12 are constructed from a material that is different from the middle portion 52. As illustrated, both the first end 10 and the second end 12 can be constructed from a different material than the middle portion 52. In an exemplary embodiment, the first end 10 and/or the second end 12 can be constructed from a material that replicates the material of the disc. Examples of suitable materials include PEEK, titanium, Medical grade varying durometer implantable polymers, polyeurethane, and Bionate.

A number of different techniques may be used for accessing the intervertebral disc space in accordance with embodiments of the present invention. For example, the intervertebral disc space may be accessed using an anterior, lateral, or posterior approach. Combinations of these approaches (e.g., posterolateral) can also be used. It should be understood, however, that an anterior approach to the disc space may pose risks to a patient's organs which may be encountered when accessing the disc space anteriorly through the patient's abdomen. While posterior or posterolateral approaches typically pose less risk of damage to the patient's organs, they can increase risk of undesirable nerve damage.

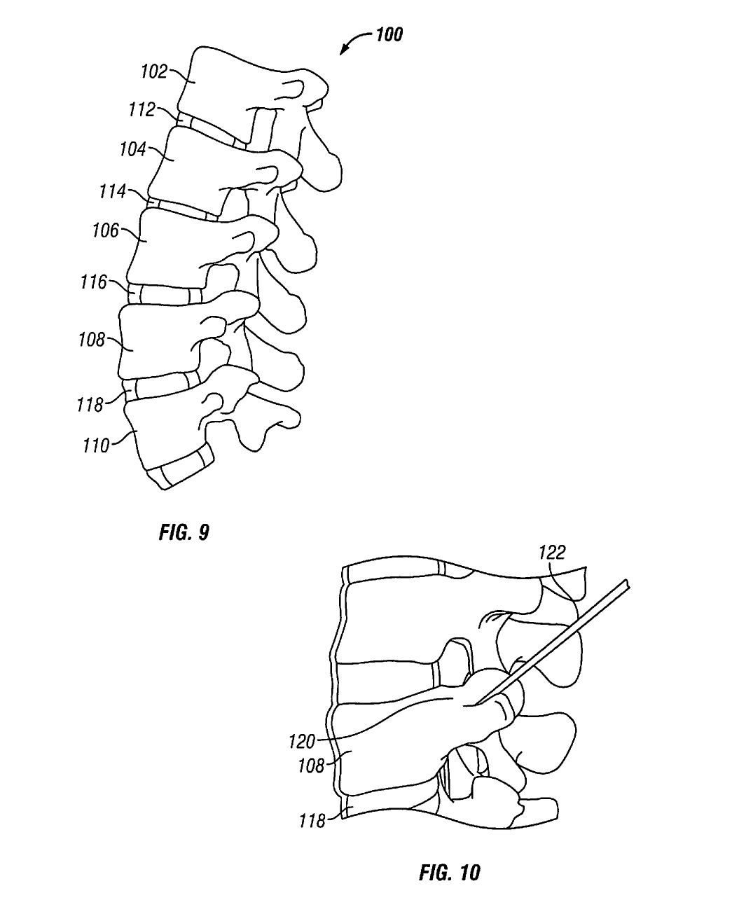

An embodiment for accessing an intervertebral disc space is now discussed with reference to FIGS. 9-14. As will be discussed in more detail, this technique utilizes a trans-pedicle-endplate approach to form an access channel to the disc space. This approach should reduce the risk to nerves and other sensitive tissues caused accessing the disc space. This approach should be particularly useful for insertion of balloons into the disc space.

FIG. 9 illustrates a spinal region 100 in which the trans-pedicle-endplate approach may be used to access the disc space in accordance with embodiments of the present invention. In an embodiment, the spinal region 100 is the lumbar region of a patient's spine. While the lumbar region is illustrated by FIG. 9, it should be understood that the trans-pedicle-endplate approach described herein may be used in other regions of the spine. In the spinal region 100, lumbar vertebra 102, 104, 106, 108, 110 are separated by four discs 112, 114, 116, 118.

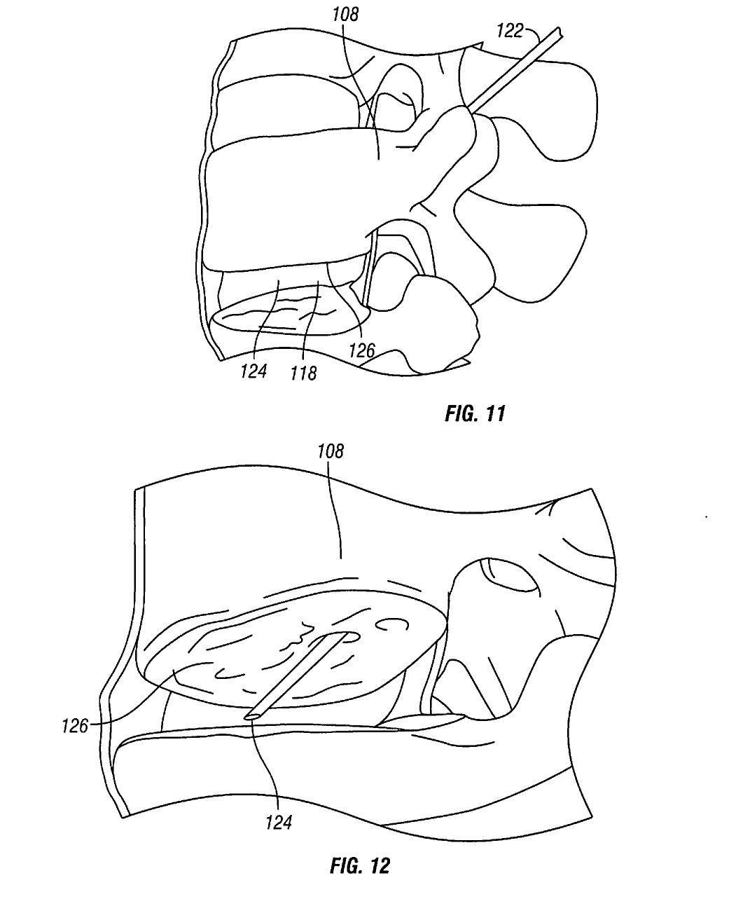

FIGS. 10-12 illustrate creation of an access channel to the disc 118 through pedicle 120 of superior vertebra 108. To create the access channel, a drill 122 may be inserted into the pedicle 120 of the superior vertebra 108. In an embodiment, the physician may apply longitudinal force to the drill 122 while rotating the handle (not illustrated) to force the drill through the vertebra 108 to the disc 118. As best seen in FIGS. 11 and 12, the distal end 124 of the drill 122 penetrates endplate 126 of the superior vertebra 108 to access the disc 118. In the illustrated embodiment, the endplate 126 is penetrated posteriorly. To properly access the disc 118, the drill 122 should be inserted into the pedicle 120 at a specified angle from the transverse plane. In an embodiment, the drill 122 should be inserted into the pedicle 120 at an angle of about 10° to about 60° from the transverse plane and, more preferably, about 20° to about 45° from the transverse plane. In an exemplary embodiment, the drill 122 should be inserted into the pedicle 120 at an angle of about 45°. It should be understood that the angle of approach may be from either above (as illustrated in FIGS. 10 and 11) or below the pedicle, as desired for a particular application.

Embodiments of the present technique may further include preparation of the intervertebral disc space through the access channel. For example, a diskectomy may be performed where the intervertebral disc, in its entirety, is removed. Alternatively, only a portion of the intervertebral disc can be removed. The endplates of the adjacent vertebral bodies may then scraped to create an exposed end surface for facilitating bone growth across the invertebral space. After preparation, an implant may then be inserted into the disc space.

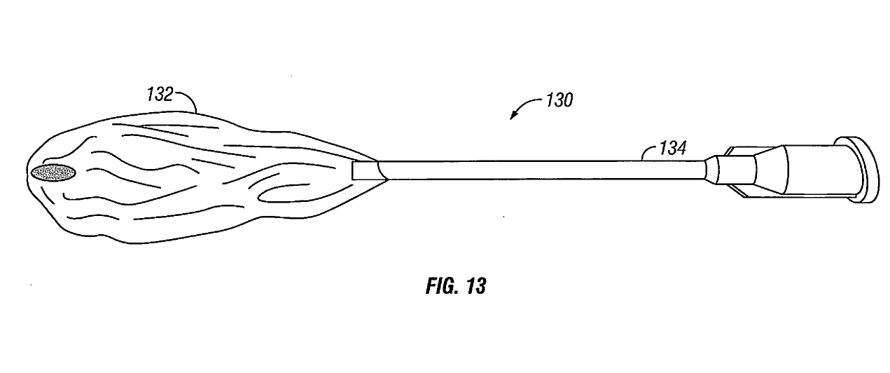

Embodiments of the present technique may further include inserting an implant (or other suitable device or apparatus) through the access channel to treat the disc 118. With reference to FIG. 13, a balloon assembly 130 is illustrated that may be inserted through the access channel in accordance with one embodiment of the present invention. In the illustrated embodiment, the balloon assembly 130 includes a balloon 132 and a rod portion 134. In an embodiment, the balloon assembly 130 may be an inflatable bone tamp. FIG. 13 illustrates the balloon 132 in a deflated state. The balloon 132 may include any of a variety of different balloons suitable for use in medical procedures. Examples of suitable balloons include those comprising plastics, composite materials, polyethylene, mylar, rubber, polyurethane, or any other suitable material. Embodiment of the invention may further include coating at least a portion of the balloon 132 with a bone growing agent. Examples of suitable bone growing agents include bone morphogenic protein, and osteoconductive bone agents.

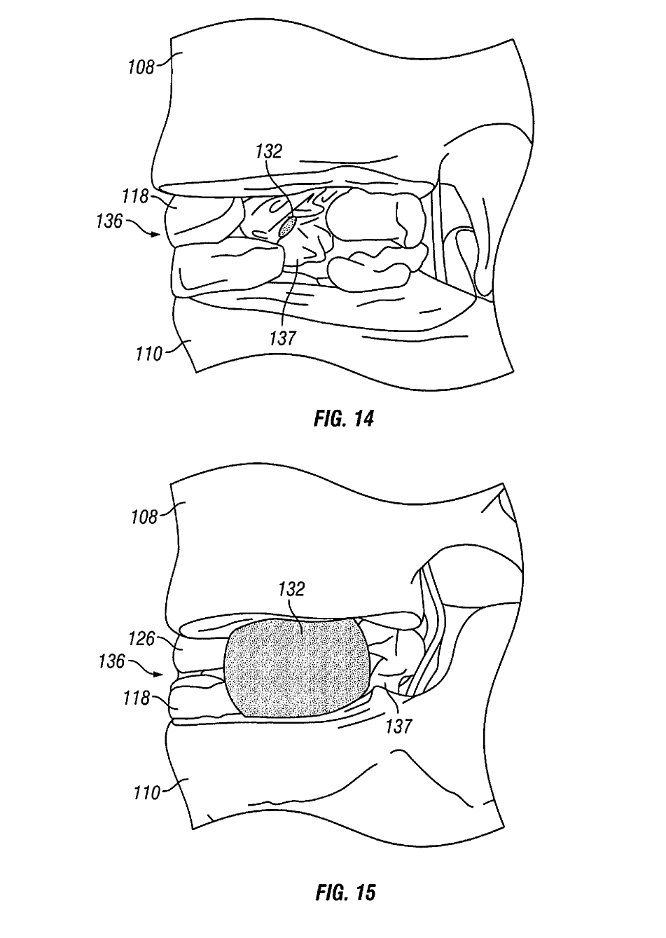

Insertion of the balloon 132 through the access channel to treat the disc 118 will now be discussed with reference to FIGS. 14 and 15. Prior to insertion of the balloon 132, the intervertebral disc space can be prepared. The intervertebral disc space is illustrated on FIG. 14 by reference number 136. Preparation of the intervertebral disc space 136 may include, for example, complete or partial removal of the disc 118. In one embodiment, at least a portion of the disc nucleus may be removed with the disc annulus remaining at least partially intact. As illustrated by FIG. 14, a cavity 137 may be formed in the disc space 136 from removal of the disc nucleus. The balloon 132 may then be inserted through the access channel and into the cavity 137 formed by removal of the disc 118. As previously discussed, the access channel has been formed through the pedicle 120 of the superior vertebra 108 penetrating the endplate 126 to provide access to the intervertebral disc space 136. As illustrated by FIG. 15, the balloon 132 may be inflated in the intervertebral disc space 136. In an embodiment, the balloon 132 may be inflated with a disc filler material. In another embodiment, the balloon 132 may be inflated with a radio-opaque contrast medium. In an exemplary embodiment, the balloon 132 may be detached from the rod portion 134, leaving the balloon 132 in the disc space 136. The filler material may cure or otherwise harden to form an implant in the disc space 136, in accordance with one embodiment. In another embodiment, the balloon 132 may be deflated and removed from the disc space 136. In an exemplary embodiment, a second balloon may then be inserted into the disc space 136 and inflated with a disc filler material. In an alternative embodiment, a the balloon 132 may be used to distract endplates of adjacent vertebral bodies for subsequent spacer insertion.

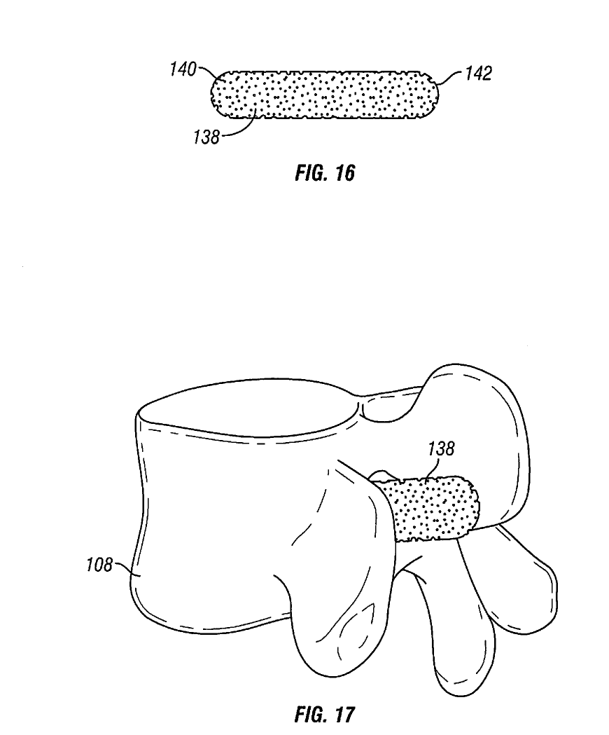

In accordance with embodiments of the present invention, an access channel may be created through the vertebra 108. FIGS. 16 and 17 illustrate a rod assembly 138 that can be used in accordance with embodiments of the present invention. Rod assembly 138 has a distal end 140 and a proximal end 142. In an embodiment, the rod assembly 138 can be used to plug the access channel. In an exemplary embodiment, the rod assembly 138 can be inserted into the access channel with the distal end 140 being inserted first followed by the proximal end 142. Any of a variety of different biocompatible materials may be used to manufacture the rod assembly 138. By way of example, suitable materials may include titanium, stainless steel, titanium alloys, non-titanium metallic alloys, polymeric materials, plastics, plastic composites, polyetheretherketone (PEEK), ceramic, and elastic materials.

The preceding description describes the use of a disc filler material in accordance with embodiments of the present invention. Those of ordinary skill in the art will appreciate that the filler material may comprise any of a variety of materials that may be utilized to, for example, fill and stabilize the intervertebral disc space. Examples of suitable materials may include bone cements (e.g. polymethyl methacrylate), human bone graft and synthetic derived bone substitutes.

Although the preceding discussion only discussed having a single implant inserted into the intervertebral disc space, it is contemplated that more than one implant can be inserted in the disc space.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. An intervertebral spacer comprising:
a body portion comprising a first end, a second end, a first side portion connecting the first end and the second end, and a second side portion connecting the first end and the second end;
wherein an entrance port is defined in the first end of the body portion;
wherein a first exit port is defined in the first side portion of the body portion; and
wherein a second exit port is defined in the second side portion of the body portion.

2. The intervertebral spacer of claim 1 wherein an upper surface of the body is rectangular in shape.

3. The intervertebral spacer of claim 1 wherein the body portion is block-shaped.

4. The intervertebral spacer of claim 1 wherein the entrance port, the first exit port, and the second exit port are in fluid communication.

5. The intervertebral spacer of claim 1 wherein the entrance port, the first exit port, and the second exit port each have a cross-sectional area of about 0.5 mm² to about 4 mm².

6. The intervertebral spacer of claim 1 wherein a ratio of a cross-sectional area of the entrance port to a surface area of the first end is less than about 1:10.

7. The intervertebral spacer of claim 1 wherein a channel formed in the body portion connects the entrance port with the first exit port and the second exit port.

8. The intervertebral spacer of claim 7 wherein a cross-sectional area of the channel is about 0.2 mm² to about 11 mm².

9. The intervertebral space of claim 7 wherein the channel proceeds from the entrance port in a direction along the longitudinal axis of the body portion, the channel dividing into at least a first branch in communication with the first exit port and a second branch in communication with the second exit port, the channel being tubular in shape.

10. The intervertebral space of claim 1 wherein the body portion further comprising a central cavity in fluid communication with the entrance port, the first exit port, and the second exit port.

11. The intervertebral spacer of claim 1 wherein the body portion is manufactured from a material comprising an elastomer.

12. The intervertebral spacer of claim 1 further comprising a plug sized for insertion into the entrance port of the body portion.

13. An intervertebral spacer comprising:
a body portion comprising:
a first end, the first end having an entrance port formed therein;
a second end;
a first side portion connecting the first end and the second end, the first side portion having a first exit port formed therein; and
a second side portion connecting the first end and the second end, the second side portion having a second exit port formed therein; and
a plug sized for insertion into the entrance port of the body portion;
wherein an upper surface of the body portion is rectangular in shape; and
wherein the entrance port, the first exit port, and the second exit port are in fluid communication.

14. A method of installing an intervertebral spacer, the method comprising:
positioning the intervertebral spacer in an intervertebral disc space, the intervertebral spacer comprising a body portion with a first end and a second end, the first end being inserted first into the disc space followed by the second end; and
flowing a disc filler material through the intervertebral spacer and into the intervertebral disc space.

15. The method of claim 14 wherein flowing the disc filler material comprises flowing the disc filler material into an entrance port in the first end of the body portion such that the disc filler material exits the body portion from at least a first exit port in a first side portion of the body portion and a second port in a second side portion of the body portion, wherein the first side portion connects the first end and the second end, and wherein the second side portion connects the first end and the second end.

16. The method of claim 14 wherein the disc filler material comprises at least one material selected from the group consisting of bone cement, human bone graft, and synthetic derived bone substitute.

17. A method of accessing an intervertebral disc space, the method comprising:
drilling an access channel to the intervertebral disc space through an adjacent vertebra, the adjacent vertebra comprising a pedicle and an endplate adjacent the intervertebral disc space, the access channel penetrating the pedicle and the endplate of the adjacent vertebra.

18. The method of claim 17 further comprising inserting a balloon into the intervertebral disc space.

19. The method of claim 18 further comprising inflating the balloon in the intervertebral disc space.

20. The method of claim 17 further comprising inserting a rod assembly into the access channel to plug the access channel.
